(19) United States
(12) Patent Application Publication
(10) Pub. No.: US 2023/0056010 A1
(43) Pub. Date: Feb. 23, 2023

(54) INTRADERMAL NEEDLE DEVICE FOR DRAWING INTERSTITIAL FLUID

(71) Applicant: X Development LLC, Mountain View, CA (US)

(72) Inventors: Anupama Thubagere Jagadeesh, Palo Alto, CA (US); Samira Darvishi, San Francisco, CA (US); Nick Robertson, Santa Clara, CA (US)

(21) Appl. No.: 17/405,782

(22) Filed: Aug. 18, 2021

(51) Int. Cl. A61B 10/00

(52) U.S. Cl. CPC A61B 10/0045; A61B 2010/008

(57) ABSTRACT

Disclosed herein is an intradermal needle device including a rigid base; a needle rigidly affixed to the rigid base, the needle including a hollow shaft extending from the rigid base along an axis to a tip of the needle; a cap formed from a deformable material affixed to the rigid base and extending along the axis, the cap comprising a cushion portion surrounding a portion of the hollow shaft, the cushion portion having a convex-shaped surface facing away from the rigid base, wherein the tip of the needle extends beyond the convex surface by a non-zero distance of 1.5 mm or less and wherein when the intradermal needle device can be pressed with the needle tip against skin of a living body, the tip pierces the skin to a depth of 1.5 mm or less and the convex-shaped surface of the cushion portion deforms against the skin surrounding the needle.

### FIELD OF THE DISCLOSURE

The disclosure relates to a device and method for drawing interstitial fluid from the dermis of a living subject.

### BACKGROUND

Dermal interstitial fluid is a thin layer of fluid surrounding the dermal cells of a subject, composed of a water solvent containing sugars, salts, fatty and amino acids, coenzymes, hormones, neurotransmitters, white blood cells and cell waste-products.

Microneedles are microscopic applicators used, for example, to deliver vaccines or other drugs across various barriers, including transdermal application. The shallow penetration depth of microneedles allow access to a region in which interstitial fluid is found in the skin, between the epidermis and dermis.

### SUMMARY

In general, the disclosure relates to an intradermal needle device including a cap composed of a soft and deformable material that, when pressed against the skin of a living subject, inserts a hollow needle into the skin. The needle penetrates the epidermis while remaining above the dermis and draws interstitial fluid found between the two layers from the subject. The deformable material of the cap presses and deforms against the skin, applying an even pressure to the contact area, and facilitating drawing of the interstitial fluid. In some examples, the cap is attached to one end of the device and is composed of a deformable material such as silicone, rubber, or other elastomers. The cap includes a convex-shaped (e.g., hemispherical) cushion portion extending from an end of the rigid base of the device. The needle protrudes from the outer surface of the cushion, oriented along the central axis of the device.

A user orients the needle of the intradermal needle device above the subject skin and presses the needle downward, thereby penetrating the skin with the needle tip, until the cushion surface contacts the skin. The distance the needle extends beyond the convex surface of the cushion corresponds to the needle penetration depth into the skin. Generally, this depth is chosen so that the needle penetrates into but not through the dermal tissue. In some examples, the needle extends by a distance of 1.5 mm or less. An opening in the needle tip creates a fluid connection allowing interstitial fluid present beneath the skin to flow into the hollow shaft of the needle. The hollow shaft of the needle becomes a channel for the interstitial fluid to flow between the user and the intradermal needle device.

The user applies pressure to the skin by pressing the intradermal needle device into the skin. The convex shape of the cushion applies a uniform pressure to the cushion-skin contact surface area, which surrounds the needle, and thereby tissue beneath. The applied pressure increases the flow rate of the interstitial fluid flowing into the hollow shaft of the needle. In some implementations, the user or other operator operates a pressure application device, such as a strap, to perform the pressing.

In some implementations, an apparatus can include several intradermal needles with the tips of the needles arranged to be substantially coplanar. Each of the intradermal needles have a cap with deformable material. The apparatus can be oriented above and pressed against the living subject, thereby penetrating the skin with the needle tips and concurrently drawing interstitial fluid into the plurality of intradermal needle devices.

In such implementations, the intradermal needle devices of the apparatus can be arranged in a two dimensional array wherein each needle tip is spaced about 20 mm or less from the tip of at least one other needle. The apparatus can include 100 or fewer intradermal needle devices (e.g., 80 or fewer, 60 or fewer, 50 or fewer, 40 or fewer, 10 or more, 20 or more).

In general, in a first aspect, the invention features an intradermal needle device including a rigid base; a needle rigidly affixed to the rigid base, the needle including a hollow shaft extending from the rigid base along an axis to a tip of the needle; a cap formed from a deformable material affixed to the rigid base and extending along the axis, the cap comprising a cushion portion surrounding a portion of the hollow shaft, the cushion portion having a convex-shaped surface facing away from the rigid base, wherein the tip of the needle extends beyond the convex surface by a non-zero distance of 1.5 mm or less and wherein when the intradermal needle device can be pressed with the needle tip against skin of a living body, the tip pierces the skin to a depth of 1.5 mm or less and the convex-shaped surface of the cushion portion deforms against the skin surrounding the needle.

Embodiments may include one or more of the following features. The device wherein the cushion portion of the cap can have an axial thickness of 15 mm or less. The cushion portion can have a maximum dimension of 30 mm or less in a direction orthogonal to the axis. The rigid base can include a first portion extending along the axis and cap comprises a sleeve that fits over an outer surface of the first portion of the rigid base extending away from a base of the cushion portion. The deformable material can be selected from the group consisting of silicone, rubber, polymers, gels, packaged liquid crystals, packaged emulsions, foams, or synthetic tissue. The deformable material can have a shore 00 durometer value in a range from 10 to 90. The tips of the needles of the intradermal needle devices can be substantially coplanar. The tip of each of the needles can be spaced about 40 mm or less from the tip of at least one other needle. The intradermal needle devices can be arranged in a two dimensional array. The apparatus can include no more than 100 intradermal needle devices (e.g., 80 or fewer, 60 or fewer, 50 or fewer, 40 or fewer, 10 or more, 20 or more). The rigid base can be sealed against liquid, or gaseous ingress.

In general, in a second aspect, the invention features a method for drawing interstitial fluid (ISF) from a living subject including inserting a needle into a skin of the living subject to a depth of 1.5 mm or less; applying pressure to skin around the needle by pressing a cushion formed from a deformable material against the skin of the living subject around the needle, the pressing being sufficient to deform a shape of the cushion to conform a surface of the cushion to the skin around the needle; and drawing from the living subject ISF through the needle while applying the pressure to the skin. Embodiments may include one or more of the following features.

In some implementations, the pressing can be performed by the living subject. The pressing can be performed by a pressure application device. The drawing can be performed by a vacuum pressure in fluid connection with the needle. The method can include a plurality of needles arranged such that tips of the needles are substantially coplanar. The drawing can terminate when a volume of drawn ISF meets a volume threshold.

In some implementations, the method can further include removing the needle from the skin of the living subject. The method can further include determining a parameter of the ISF.

Among other advantages, the system includes a method to rapidly identify subpopulations of cellular biological samples and target them with directed light. Computer vision and machine learning algorithms can be trained to identify subpopulations based on various user-selected criteria increasing system flexibility for application in a number of situations.

Additionally, the components of the microscope system are conventional imaging and detection hardware in conjunction with conventional computer vision algorithms facilitating cost-effective image collection and target identification. The use of standard components also facilitates interchangeability of components and increases the system flexibility in various implementations.

Other advantages will be apparent from the description, the drawings, and the claims.

### BRIEF DESCRIPTION OF THE DRAWINGS

FIG. 1 is a perspective schematic view of an ISF drawing apparatus including a single intradermal needle device arranged within a housing.

FIGS. 2A and 2B are schematic cross-section illustrations of the rigid base and cap of an intradermal needle device.

FIG. 2C is a schematic illustration of the cap and rigid base assembled into an intradermal needle device.

FIG. 3 is a perspective schematic cross-section illustration of the ISF drawing apparatus.

FIGS. 4A and 4B are schematic illustrations depicting the use of the intradermal needle device to draw ISF from a living subject.

FIG. 5A is a perspective schematic illustration of an intradermal needle device including an array of needles.

FIG. 5B is a perspective schematic cross-section illustration of the intradermal needle device of FIG. 5A.

FIG. 6 is a flow chart diagram depicting the steps for drawing ISF from a living subject with the intradermal needle device.

In the figures, like symbols indicate like elements.

### DETAILED DESCRIPTION

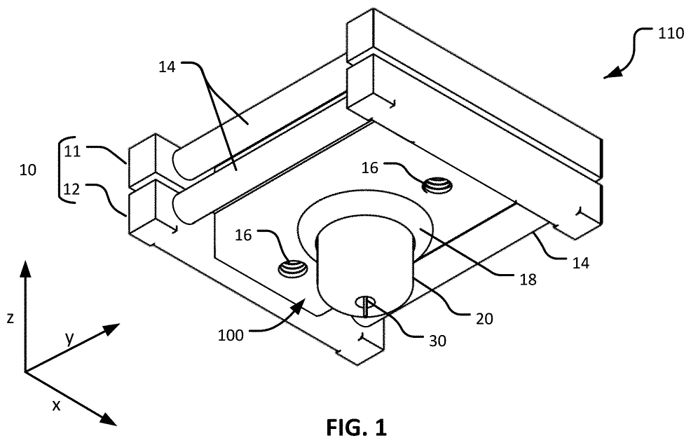

FIG. 1 is a perspective view of an intradermal needle device 100 arranged within a housing 10 forming an interstitial fluid (ISF) drawing apparatus 110. The intradermal needle device 100 includes a rigid base, a cap 20 covering a portion of the rigid base, and a needle 30 extending from the rigid base through the cap 20. The ISF drawing apparatus 110 form factor is compatible with use on a living subject and, in some implementations, capable of manipulation by a single hand for self-application by the subject. The housing 10 is composed of an upper housing 11 and a lower housing 12 which are arranged to enclose and seal the intradermal needle device 100 against liquid egress. The lower housing 12 includes a collar 18 which orients the intradermal needle device 100 in the housing 10 and maintains the intradermal needle device 100 position during application to the subject skin and provides a surface on which the upper housing 11 and lower housing 12 apply a compression force on the intradermal needle device 100 when compressed together.

The housing 10 external dimensions can be in a range from 10 mm to 50 mm wide, 10 mm to 50 mm long, and 3 mm to 10 mm tall when assembled. The depicted housing 10 is square though other shapes are possible, such as circular, rectangular, or ovoid.

The housing 10 material is rigid under an applied load sufficient to pierce the skin and can include smooth surfaces to facilitate easy cleaning and sanitation. For example, the material is a rigid plastic, such as high-density polyethylene (HDPE), or polypropylene (PP), or a biocompatible metal or metallic alloy, such as stainless steel. Materials with high melting temperatures convey durability to steam- or pressure-sterilization facilitating reusability and routine use, for example, in a medical care setting.

In some implementations, the upper housing 11 and lower housing 12 are of unitary construction (e.g., 3D printed). Alternatively, the upper housing 11 and lower housing 12 are assembled from individually constructed components.

The ISF drawing apparatus 110 includes a single intradermal needle device 100, though in alternative implementations, the ISF drawing apparatus 110 can include more than one intradermal needle device 100. In some implementations, the ISF drawing apparatus 110 can include up to (e.g., no more than) 100 intradermal needle devices 100 (e.g., 80 or fewer, 60 or fewer, 50 or fewer, 40 or fewer, 10 or more, 20 or more).

The cap 20 is composed of a deformable and elastic material such as silicone, rubber, polymers, gels, packaged liquid crystals, packaged emulsions, foams, or synthetic tissue. In some implementations, the cap 20 has a shore 00 durometer value in a range from 10 to 90 (e.g., 90 or less, 70 or less, 50 or less, 10 or more, or 30 or more). In alternative implementations, the cap 20 has a shore A durometer value in a range from 0 to 60 (e.g., 60 or less, 40 or less, 30 or less, 0 or more, 10 or more, or 20 or more). Lower durometer values provide increased deformability when the cap 20 presses against the subject skin and increases subject comfort during ISF drawing.

The needle 30 is a hollow tube extending along the central longitudinal axis from the rigid base. The needle 30 has a gauge of 25 or lower (e.g., 27 or lower, 30 or lower, 32 gauge or lower, or 34 gauge) with lower gauge reducing pain potential in the living subject during needle 30 insertion into the dermis and higher gauges increasing the ISF collection rate. The needle 30 extends from the rigid base by a distance in a range from 20 mm or less (e.g., 18 mm or less, 15 mm or less, 12 mm or less, 5 mm or more, 8 mm or more, or 10 mm or more) and terminates in a beveled tip 32. The needle 30 lumen terminates at the tip 32 and in alternative implementations, the lumen terminates at a side vent of the needle 30 (e.g., a side-vented needle).

The needle 30 extends from the cushion portion 22 by a distance that facilitates collection of ISF by piercing the epidermis of a subject without penetrating the dermis. The distance can vary based on one or more physical parameters of the subject, or one or more ISF collection parameter such as subject age, weight, race, sex, existing medical condition, diagnostic result, desired diagnostic sample, or preferred ISF collection volume. In some implementations, the needle 30 extends from the cushion portion 22 by a distance in a range from 100 μm to 1500 μm (e.g., 100 μm to 1500 μm, 100 μm to 1000 μm, 100 μm to 800 μm, 100 μm to 500 μm, 500 μm to 1500 μm, 1000 μm to 1500 μm, 200 μm to 800 μm, or 400 μm to 1000 μm).

The upper housing 11 and lower housing 12 include two cylindrical bars 14 on opposing ends that when aligned provide a force-application area for a pressure application device, e.g., a strap, for the ISF drawing apparatus 110. In some implementations, the ISF drawing apparatus 110 includes a binding feature that connects the bars 14 and allows temporary constraint to the subject when the ISF drawing apparatus 110 is applied to the epidermis (e.g., skin) of the subject. For example, the binding feature can include one or more straps that bind the upper and lower bars 14 together and encircle the subject to maintain the ISF drawing apparatus 110 position and orientation. The binding feature applies a force to the ISF drawing apparatus 110 which holds the ISF drawing apparatus 110 in place against the subject epidermis.

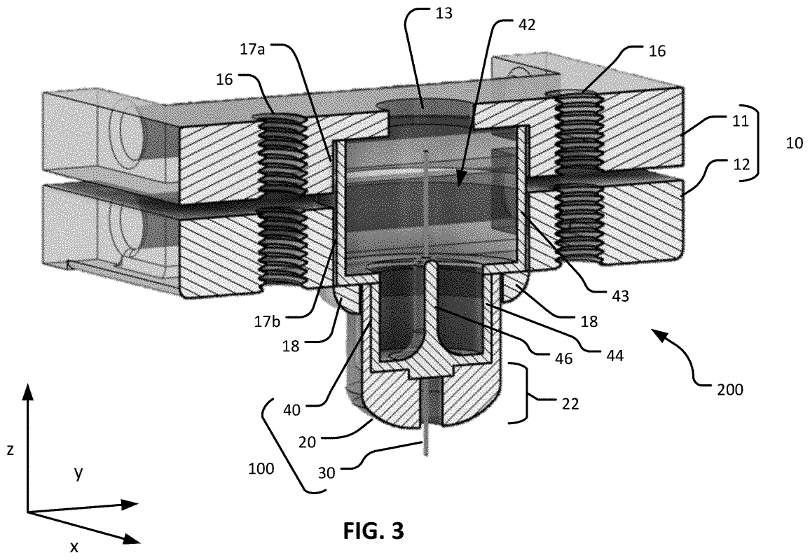

The upper housing 11 and lower housing 12 include a reversible sealing mechanism which, when utilized, tighten the upper housing 11 and lower housing 12 together and seals the rigid base 40 against liquid egress. The housing 10 of FIGS. 1 and 3 includes two threaded holes 16 for receiving a threaded screw. When the housing 10 is formed around the intradermal needle device 100 the holes 16 in the upper housing 11 and lower housing 12 are aligned. A screw threaded between the holes 16 of the upper housing 11 and lower housing 12 draws them together and applies a compressive force to the rigid base 40, thereby maintaining the position and orientation of the intradermal needle device 100 within the housing 10. To disassemble the housing 10, the threaded screws are removed from the holes 16 and the upper housing 11 and lower housing 12 separate.

In an alternative implementation, the upper housing 11 or the lower housing 12 include locking tabs which couple to openings on the opposing member. The upper housing 11 and lower housing 12 are moved together to form the housing 10. The locking tabs pass through corresponding openings in the opposing member and positively engage at least a portion of the opening perimeters, thereby reversibly coupling the housing 10. To disassemble the housing 10, the locking tabs can be moved away from the opening perimeters to disengage with the openings.

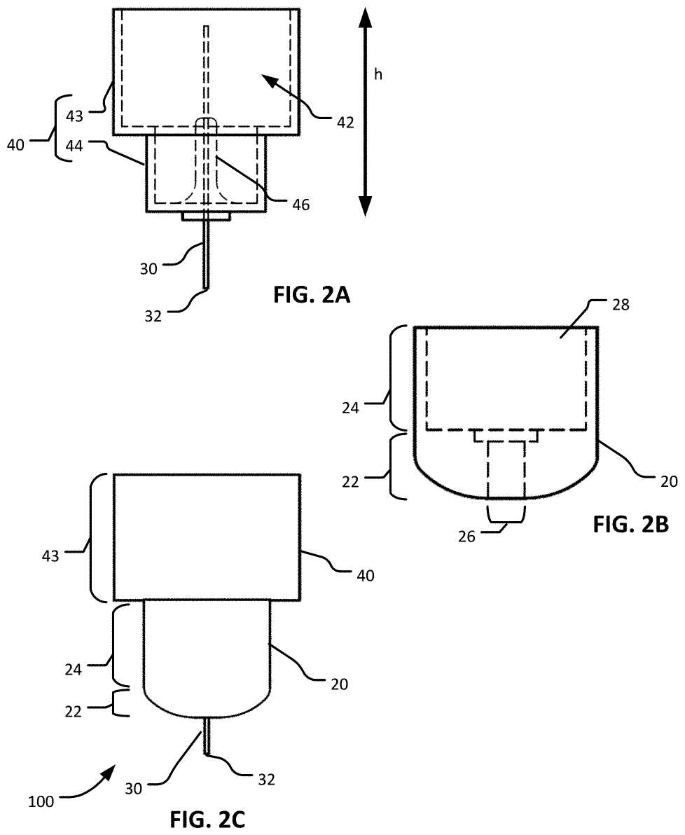

Referring to FIG. 2A through 2C, the components of the intradermal needle device 100 are shown including the cap 20, needle 30, and rigid base 40. FIG. 2A is a cross-sectional schematic illustration of the rigid base 40 and the needle 30 along a plane bisecting the rigid base 40 central axis of symmetry. In some implementations, the rigid base 40 is a commercially-available needle device, such as a Micro-Fine™, Care-Fine™, or Ultra-Fine™ rigid based manufactured by BD (Franklin Lakes, N.J.).

The rigid base 40 constitutes an upper chamber 43 and a lower chamber 44 collectively having an inner volume 42. The upper chamber 43 and lower chamber 44 are cylindrical and each having a respective inner (ID) and outer (OD) diameter. The height of the rigid base 40 is sufficient to fully encapsulate the needle 30 portion extending into the rigid base 40 and in some implementations is in a range from 2 mm to 20 mm (e.g., 5 mm to 20 mm, 10 mm to 20 mm, or 5 mm to 15 mm). Larger rigid base 40 heights accommodate higher ISF collection volumes and higher aspect ratios (e.g., higher height to rigid base 40 maximum OD ratios) for smaller intradermal needle device 100 pitches with the ISF drawing apparatus 110. The inner volume 42 receives ISF from the needle 30 end arranged within the inner volume 42 when the needle 30 is inserted into the epidermis of the living subject and ISF is collected through the needle 30 lumen. In some implementations, the inner volume 42 is evacuated creating a vacuum before the intradermal needle device 100 is installed in the housing 10 and increasing the ISF collection rate.

In alternative implementations, the rigid base 40 has a polygonal cross-sectional shape such as a rectangle, pentagon, or hexagon. In some implementations, the upper chamber 43 and lower chamber 44 have different cross-sectional shapes, such as the upper chamber 43 being cylindrical and the lower chamber 44 being polygonal, or vice versa. A rigid base 40 having a polygonal shape and a housing 10 constructed to receive the polygonal rigid base 40 can have increased rotational stability around the needle 30 axis during application to the living subject. An ISF drawing apparatus 110 including more than one polygonal rigid base 40 increases intradermal needle device 100 packing efficiency and total received ISF volume for similar pitch compared to cylindrical intradermal needle devices 100.

The upper chamber 43 OD is in a range from 3 mm to 20 mm with lower OD values facilitation higher intradermal needle device 100 densities (e.g., lower intradermal needle device 100 pitch) in ISF drawing apparatus 110 implementations having more than one intradermal needle device 100. The upper chamber 43 OD is greater than the lower chamber 44 OD which is in a range from 2 mm to 18 mm with higher values (e.g., 5 mm or more) having greater internal volumes for ISF collection.

The rigid base 40 height is the combined height of the upper chamber 43 and lower chamber 44 and can be in range from 10 mm to 30 mm with lower heights enabling lower housing 10 profiles for ease of application. The height ratio between the upper chamber 43 and lower chamber 44 is approximately equal but in alternative implementations, the upper chamber 43 can be longer than the lower chamber 44 to increase the The needle 30 is encased by a support 46 for a portion of the needle 30 length (e.g., at least the lower chamber 44 height) within the inner volume 42 and terminates within the upper chamber 42 volume. The support 46 provides increased positional stability and translates force applied to the housing 10 and rigid base 40 to the needle 30 during application.

FIG. 2B is a cross-sectional schematic illustration of the cap 20 which includes a cushion portion 22 and a collar portion 24. The cushion portion 22 is a convex-shaped surface facing away from the rigid base 40. The cushion portion 22 extends from the interior volume 28 along the needle 30 axis by a distance (e.g., an axial thickness) which surrounds the needle 30 extending from the rigid base 40. The cushion portion 22 can extend by a distance of 8 mm or less (e.g., 7 mm or less, 6 mm or less, 5 mm or less, 4 mm or less, 1 mm or more, 2 mm or more, 3 mm or more) with lower distances (e.g., 4 mm or less) providing less cushion support than larger distances (e.g., more than 4 mm). The distance that the cushion portion 22 extends from the rigid base 40 corresponds with the distance the needle 30 extends from the bottom surface of the rigid base 40. Lower needle 30 extension distances correspond with lower cushion portion 22 axial thicknesses such that the needle 30 extension distance is greater than the cushion portion 22 axial thickness.

The cushion portion 22 and the collar portion 24 have a common maximum dimension (e.g., cap 20 OD) in a direction orthogonal to the needle 30 axis, though in some implementations, the cushion portion 22 and collar portion 24 have different dimensions. Cap 20 OD values of 15 mm or less (e.g., 12 mm or less, 10 mm or less, 9 mm or less, 8 mm or less, 7 mm or less, 6 mm or less, 2 mm or more, 3 mm or more, 4 mm or more, 5 mm or more) maintains a sufficiently low contact area with the subject skin which provides pressure around the needle 30 sufficient to increase the ISF collection rate when force is applied to the ISF drawing apparatus 110. The cap 20 OD corresponds to the distance the needle 30 extends from the rigid base 40 or, alternatively, the distance the needle 30 extends from the cushion portion 22. For example, the cap 20 OD is 8 mm if the needle 30 extends from the cushion portion 22 by 1 mm.

The cushion portion 22 includes a cylindrical central hollow 26 extending from the convex-shaped surface to the collar portion 24 interior volume 28. The hollow 26 surrounds a portion of the needle 30 length extending from the lower chamber 44. The hollow 26 ID is greater than the needle 30 OD and less than the collar portion 24 ID. For example, the hollow 26 ID is larger than the needle 30 OD by 1 mm or more (e.g., 1 mm or more, 1.5 mm or more, 2 mm or more). The hollow 26 ID is beneficially within 2 mm of the needle 30 OD to provide sufficient compressive force to the area surrounding the insertion point of the living subject skin.

The cylindrical collar portion 24 has a larger OD than the lower chamber 44 and includes an interior volume 28 having a shape which substantially matches the outer shape of the rigid lower chamber 44 (e.g., cylindrical). For example, the interior volume 28 has an ID that is within 0.1 mm of the lower chamber 44 OD such that the collar portion 24 removably affixes to the lower chamber 44 through static friction force (e.g., a pressure fit). FIG. 2C is a schematic illustration of the collar portion 24 of the cap 20 affixed to the lower chamber 44 of the rigid base 40 and constituting an intradermal needle device 100.

FIG. 3 is a cross-section view along the y-axis (inset) of the ISF drawing apparatus 110 of FIG. 1 and shows the arrangement of the intradermal needle device 100 within the housing 10, e.g., between the upper housing 11 and lower housing 12. The intradermal needle device 100 includes the cap 20 and needle 30 shown externally in FIG. 1A, and the rigid base 40 enclosed and sealed within the housing 10.

The upper housing 11 includes recession 17a and the lower housing 12 includes recession 17b such that when the upper housing 11 and lower housing 12 are aligned and arranged into the housing 10, the combined recessions, collectively recessions 17, and the collar 18 enclose and seal the intradermal needle device 100 within the housing 10. The diameter of the recessions 17 is approximately equal (e.g., within 1 mm) to the OD of the intradermal needle device 100 arranged within the housing 10. The combined height of the recessions 17 is less than or equal to the total height of the rigid base 40 such that when the reversible sealing mechanism is actuated (e.g., screws are threaded into the holes 16) the housing 10 compresses the rigid base 40 within the recessions 17.

The upper housing 11 includes an optional window 13 in which a transparent material, e.g., glass, polypropylene, polyvinyl chloride, or polycarbonate, is affixed, through which the contents of the intradermal needle device 100 are viewed. The window 13 has a smaller diameter than the upper chamber 43 such that the upper housing 11 material overlaps the upper chamber 43 walls.

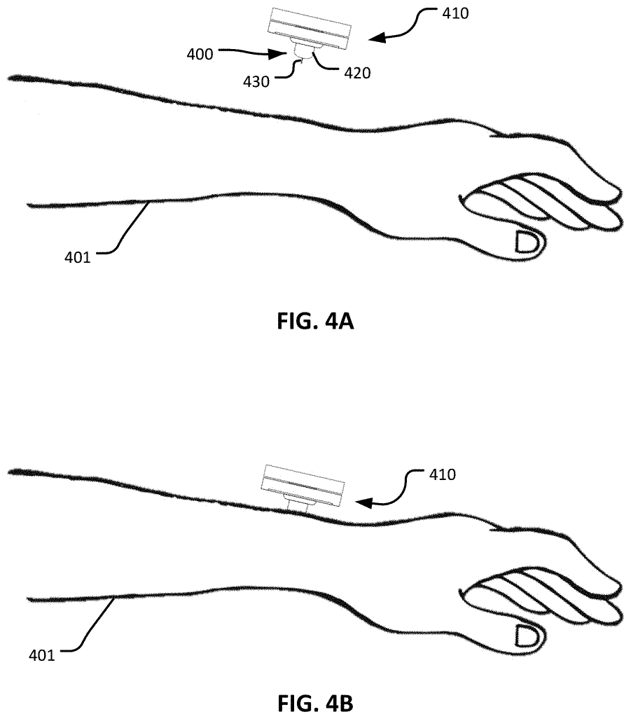

The needle 30 extends from the cushion portion 22 such that when the ISF drawing apparatus 110 is pressed against the epidermis of the subject, the needle 30 penetrates to a depth and is arrested when the cushion portion 22 is compressed against the subject. FIGS. 4A and 4B depict the use of an exemplary ISF drawing apparatus 400 to draw ISF from a subject forearm 401. FIGS. 4A and 4B depict a subject forearm 401 but the ISF drawing apparatus 400 can draw ISF from any area of the body in which the epidermis is accessible, such as the wrist, bicep, shoulder, trapezius, torso, belly, thigh, calf, or hip. Applying the ISF drawing apparatus 400 to an area of the epidermis situated above a major muscle or fat group can reduce subject discomfort by distributing the pressure more evenly and avoiding hard surfaces beneath the needle 430. In FIG. 4A, the ISF drawing apparatus 400 is oriented above the including the cap 420 and needle 430 extending from the ISF drawing apparatus 400 surface facing the epidermis of the depicted subject arm 410.

The ISF drawing apparatus 400 is used to collect ISF occupying the space between the outermost two layers of skin, the dermis and epidermis of a user. The epidermis is the outermost layer of cells layered above the dermis of a user, the dermis being a layer of tissue, containing blood capillaries, nerve endings, sweat glands, a base layer of columnar cells (e.g., the stratum basale), and other structures. Capillaries are semi-permeable barriers carrying and exchanging a number of biological compounds around the body, including to and from the dermis. These compounds commonly include oxygen, nutrients, metabolic waste, and biomarkers from other areas of the body.

The extracellular ISF found outside of the capillaries perfuses the region between the layers of the epidermis and the underlying cells of the base layer. The ISF drawing apparatus 400 applied to the skin of a user penetrates the epidermis and provides a means to draw ISF without disrupting nerve endings within the base layer of the dermis, thereby avoiding an induced pain response for the user.

As shown in FIG. 4B, the ISF drawing apparatus 410 is applied to the forearm 401 and the needle 430 punctures the epidermis. The needle 430 tip enters the area above the base layer of the dermis containing the ISF. The central channel of the needle 430 draws a sample of the ISF into the inner chamber, e.g., inner volume 42. The pressure applied to the ISF drawing apparatus 410 normal to the forearm 401 epidermis increases the ISF flow. In some implementations, reduced pressure in the inner volume 42 further draws the sample of the ISF into the inner volume 42. In some implementations, a capillary tube is connected to the exposed end of the needle 30 and sealed against support 46. The ISF is drawn through the capillary tube into an external receiving vessel for collection. Such implementations can reduce the potential for contamination of the ISF during collection.

In some implementations, the intradermal needle device 100 can include more needle 30 wherein the tips 32 are coplanar and extend from the cap 20 by a uniform distance. ISF drawing apparatus 110 implementations including these configurations can provide higher ISF drawing speeds and/or draw ISF into individual rigid bases 40 with respective isolated inner volumes 42 to collect multiple ISF samples in parallel.

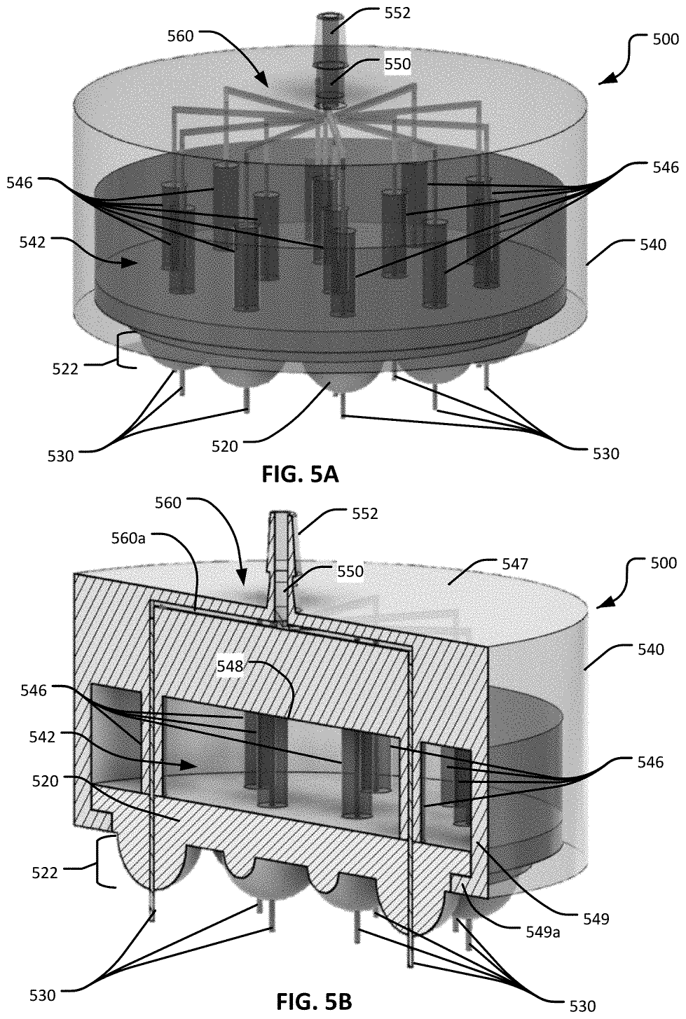

FIGS. 5A through 5C show an intradermal needle device 500 including a unitary rigid base 540 supporting a cap 520 having multiple cushion portions 522 and a needle array 530 of fourteen needles, such as the needle 30 of the intradermal needle device 100, connected through a common liquid connection system 560 to a liquid vent 550. The vent 550 extends from the intradermal needle device 500 upper surface 547 opposite the needle array 530. Referring to FIGS. 5A and 5B, a perspective view of the ISF drawing apparatus 510 and a cross-sectional view through the central vent 550 axis are shown, respectively.

The rigid body 540 is a cylindrical unitary body which encloses the connection system 560. The vent 550 extends from the upper surface 547 while the support array 546 extends away from the lower surface 548. The rigid body 540 includes an inner volume 542 defined by the lower surface 548 and the inner surface of the outer wall 549 extending from the along the circumference of the intradermal needle device 500. The support array 546 extends from the lower surface 548 through the inner volume 542 by a distance and each support of the support array 546 encloses a needle of the needle array 530 by a portion of the needle length. The needle array 530 extends from the support array 546 by a distance and are partially enclosed by the cap 520.

The cap 520 is composed of the low durometer material described above and includes a cushion portion 522 for each needle of the needle array 530. The needle array 530 extends from the cushion portion 522 by a distance which penetrates the subject epidermis when the intradermal needle device 500 is applied to the skin of a living subject. The cap 520 is supported around the circumference by a lip 549a which extends inward from the outer wall 549 by a small distance which does not interfere with any cushion portion 522. The needle array 530 extends through the cap 520 which is in contact with the enclosed length of the needle array 530, alternative to the hollow 26 of the cap 20 of the intradermal needle device 100. The cap 520 being in contact with the needle array 530 provides increased support to the needle array 530 when applied to the living subject.

The connection system 560 is a branched liquid connection array connecting the respective lumens of the needle array 530 to the vent 550. Each branch terminates at a needle of the needle array 530, such as for example branch 560a terminating at the upper end of needle When the needle array 530 pierces the epidermis, the ISF flows through the respective lumens of the needle array 530 and through the connection system 560 which terminates at the vent 550. The vent 550 terminates in a flanged end 552 which facilitates temporary connection to additional components in the collection of ISF from a living subject, such as liquid tubing, or syringes. In some implementations, the flanged end 552 is a Luer connection.

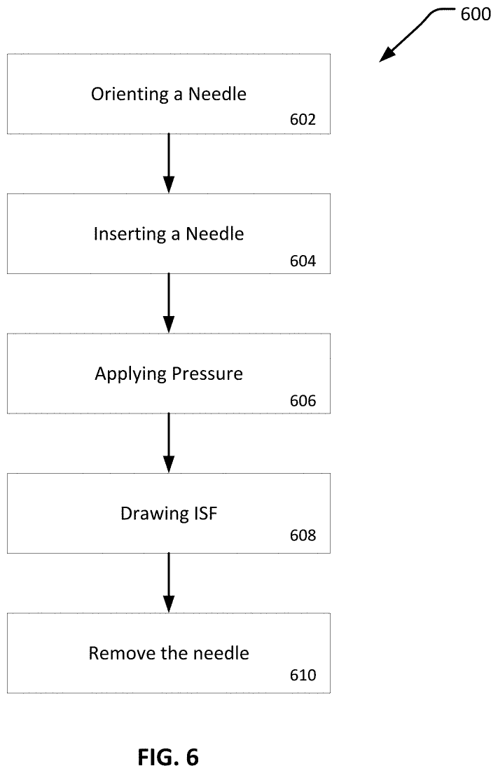

FIG. 6 is a flow-chart diagram depicting the steps for a method for drawing ISF from a living subject. An ISF drawing apparatus 110 including one or more intradermal needle device 100 is oriented above the epidermis of a living subject (step 602). Each of the intradermal needle device 100 includes a needle 30 connecting to an inner volume 42 of the intradermal needle device 100. The ISF drawing apparatus 110 is oriented such that the needle 30 is above the epidermis and oriented approximately perpendicular to the epidermis surface (e.g., within ±15° from perpendicular). In implementations in which the ISF drawing apparatus 110 includes more than one intradermal needle device 100, the tip 32 of each needle 30 is coplanar and the ISF drawing apparatus 110 can be oriented such that the plane of the tips 32 is approximately parallel (e.g., within ±15° from parallel) with the epidermal surface plane.

The ISF drawing apparatus 110 is brought toward the living subject epidermis such that the one or more needle 30 are inserted into the subject skin to a depth. The ISF flows between the epidermal and dermal skin layers and the needle 30 penetrates to a depth of less than 1.5 mm to draw the ISF from the living subject (step 604). The needle 30 penetrates until the epidermis contacts the cap 20 composed of a deformable material affixed to the rigid base 40 of the intradermal needle device 100.

Pressing the ISF drawing apparatus 110 toward the epidermis applies pressure to the skin around the needle 30 as the cushion portion 22 of the cap 20 deforms to conform to the surface of the skin (step 606). The cushion portion 22 being of low durometer conforms to the epidermis around the needle 30. The cushion portion 22 distributes the pressure from the ISF drawing apparatus 110 to the area surrounding each associated needle 30.

ISF draws into the intradermal needle device 100 while the tip 32 of the needle 30 is within the subject epidermis (step 608). Applying pressure to the ISF drawing apparatus 110 increases the ISF collection rate from the subject. In some implementations, the drawn ISF is collected within the inner volume 42 of the intradermal needle device 100, or in alternative implementations, is drawn out from the intradermal needle device 100 by a capillary tube connected to the exposed end of the needle 30 and sealed against support 46. In implementations including a vent, such as the vent 550 of intradermal needle device 500, a capillary tube can be connected to the vent and ISF drawn through the capillary tube. In some implementations, evacuating the inner volume 42, e.g., reducing the gas pressure within the inner volume 42, increases the ISF collection rate. In alternative implementations, negative pressure is applied to the end of the needle 30 opposite the user through a capillary tube to increase the ISF collection rate. In yet more alternative implementations, positive pressure is applied to the subject epidermis to increase the ISF collection rate.

The ISF drawing apparatus 110 can be in contact with the subject epidermis and the tip 32 of the needle 30 positioned between the layers of the dermis and epidermis for a time period in which a volume of ISF is drawn. The drawn ISF volume can be sufficient to fill the inner volume 42 to a level, to collect a set quantity of ISF, or in implementations in which the ISF is drawn through a vent, until a total volume is drawn through the intradermal needle device 100, such as intradermal needle device 500. For example, the drawn ISF volume can be in a range from 1 μL to 10 mL. The drawing can terminate when the volume extracted is balanced with the pressure difference between pressure applied on the skin and atmospheric pressure.

Bringing the ISF drawing apparatus 110 out of contact with the subject epidermis removes the needle 30 from the subject epidermis (step 610) and terminates drawing of the ISF. The needle 30 can be removed based on the volume of ISF collected, or total insertion time, or other relevant collection parameters such as subject discomfort or pain response.

A number of implementations have been described. Other implementations are in the following claims.

What is claimed is:

1. An intradermal needle device, comprising:
a rigid base;
a needle rigidly affixed to the rigid base, the needle comprising a hollow shaft extending from the rigid base along an axis to a tip of the needle; and
a cap formed from a deformable material affixed to the rigid base and extending along the axis, the cap comprising a cushion portion surrounding a portion of the hollow shaft, the cushion portion having a convex-shaped surface facing away from the rigid base,
wherein the tip of the needle extends beyond the convex-shaped surface by a non-zero distance of 1.5 mm or less and wherein when the intradermal needle device is pressed with the tip of the needle against skin of a living body, the tip pierces the skin to a depth of 1.5 mm or less and the convex-shaped surface of the cushion portion deforms against the skin surrounding the needle.

2. The device of claim 1, wherein the cushion portion of the cap has an axial thickness of 15 mm or less.

3. The device of claim 1, wherein the cushion portion has a maximum dimension of 30 mm or less in a direction orthogonal to the axis.

4. The device of claim 1, wherein the rigid base comprises a first portion extending along the axis and cap comprises a sleeve that fits over an outer surface of the first portion of the rigid base extending away from a base of the cushion portion.

5. The device of claim 1, wherein the deformable material is selected from the group consisting of silicone, rubber, polymers, gels, packaged liquid crystals, packaged emulsions, foams, and synthetic tissue.

6. The device of claim 1, wherein the deformable material has a shore 00 durometer value in a range from 10 to 90.

7. The device of claim 1, wherein the rigid base is sealed against liquid, or gaseous ingress.

8. An apparatus for drawing interstitial fluid from a living subject, comprising a plurality of intradermal needle devices according to claim 1, wherein the tips of the needles of the intradermal needle devices are substantially coplanar.

9. The apparatus of claim 8, wherein the tip of each of the needles are spaced about 40 mm or less from the tip of at least one other needle.

10. The apparatus of claim 8, wherein the intradermal needle devices are arranged in a two dimensional array.

11. The apparatus of claim 8, wherein the apparatus comprises no more than 100 intradermal needle devices.

12. A method for drawing interstitial fluid (ISF) from a living subject, comprising:
inserting a needle into a skin of the living subject to a depth of 1.5 mm or less;
applying pressure to skin around the needle by pressing a cushion formed from a deformable material against the skin of the living subject around the needle, the pressing being sufficient to deform a shape of the cushion to conform a surface of the cushion to the skin around the needle; and
drawing from the living subject ISF through the needle while applying the pressure to the skin.

13. The method of claim 12, wherein the pressing is performed by the living subject.

14. The method of claim 12, wherein the pressing is performed by a pressure application device.

15. The method of claim 12, wherein the drawing is performed by a vacuum pressure in fluid connection with the needle.

16. The method of claim 12, wherein the method includes a plurality of needles arranged such that tips of the needles are substantially coplanar.

17. The method of claim 12, wherein the drawing terminates when a volume of drawn ISF meets a volume threshold.

18. The method of claim 12, further comprising removing the needle from the skin of the living subject.

19. The method of claim 12, further comprising determining a parameter of the ISF.

20. The method of claim 17, wherein the volume threshold is in a range from 1 μL to 10 mL.
